(19) United States
(12) Patent Application Publication
(10) Pub. No.: US 2010/0312186 A1
(43) Pub. Date: Dec. 9, 2010

(54) SOFT TISSUE DISSECTOR

(75) Inventors: Rachana S. Suchdev, Hollis, NH (US); David L. Regan, Pelham, NH (US); Stephen M. Martone, Nashua, NH (US); Nilendu Srivastava, Hollis, NH (US); Gary Douglas, Billerica, MA (US)

Correspondence Address: Vern Maine & Associates, 547 AMHERST STREET, 3RD FLOOR, NASHUA, NH, 03063-4000, US

(73) Assignee: Vascular Technology Inc., Nashua, NH (US)

(21) Appl. No.: 12/797,067

(22) Filed: Jun. 9, 2010

Related U.S. Application Data
(60) Provisional application No. 61/185,348, filed on Jun. 9, 2009.

(51) Int. Cl. A61M 37/00; A61B 17/00

(52) U.S. Cl. 604/131; 606/190

(57) ABSTRACT

A system for the dissection of soft tissue is provided, the system comprising: a dissection tip, the tip having a rigid, non-absorptive, end shell; the rigid end shell having a textured exterior surface configured to purchase soft tissue and having at least one porosity; and the dissection tip being configured to be coupled to a suction source.

### RELATED APPLICATIONS

This application claims the benefit of U.S. Provisional Application No. 61/185,348, filed Jun. 9, 2010. This application is herein incorporated by reference in its entirety for all purposes.

### FIELD OF THE INVENTION

The invention relates to soft tissue dissectors, and more particularly, to a soft tissue dissector with suction/irrigation capability configured for use with known suction/irrigation probes.

### BACKGROUND OF THE INVENTION

Minimally invasive procedures have established a strong foothold in the realm of medicine. The advent of “scopes” (ex. endoscopes, laparoscopes) has allowed physicians to gain access to body chambers/potential spaces and perform therapeutic procedures which could once only be achieved in an open surgical fashion. The advantage to most minimally invasive procedures is decreased morbidity for the patient, often leading to decreased recovery times and decreased pain.

However, minimally invasive procedures can often be technically challenging in many regards. One such challenge lies in the introduction and/or exchange of surgical tools through small ports in minimally invasive procedures—these require greater time and dexterity than that required in open surgical procedures. To overcome this obstacle, surgeons may use one instrument to perform multiple functions during minimally invasive procedures. One such common practice in laparoscopic urology is to use a suction/irrigation probe to perform blunt tissue dissection. While a suction/irrigation probe has the strength and rigidity to allow surgeons to sweep away layers of soft tissue during dissection, it does not possess a tip that is ideally suited for fine soft tissue dissection. Such dissection is typically achieved using a Kittner instrument. A suction/irrigation probe may also be used to provide temporary hemostasis, by allowing a surgeon to place pressure on a bleeding vessel with the probe. However these probes are not indicated for such use. Also, the tip of a suction/irrigation probe is not ideally suited for such a function. Furthermore, a suction/irrigation probe may be used for retraction during minimally invasive procedures. The tip of current suction/irrigation probes again does not provide for an atraumatic, frictional surface that would be ideal for retraction. Finally, current suction/irrigation probes have several design limitations that become problematic with clinical use. For example, current probes often become obstructed with blood clots during suctioning. Thus, what is needed is an improved means by which surgeons can perform atraumatic soft tissue dissection, apply temporary hemostasis, and apply retraction, while simultaneously providing suction and/or irrigation capability. However, several obstacles must be overcome to design such a device.

First, Kittners for minimally invasive procedures have traditionally utilized cotton or gauze tips that provide absorbency and allow for non-traumatic soft tissue dissection. Such Kittners easily lose their shape and strength as they become saturated with fluids, and may shed fibers within the surgical field. Thus, traditional Kittners need to be replaced frequently during minimally invasive procedures. While an absorbent tip is beneficial in open procedures, such absorbency is less important in laparoscopic procedures which are performed under pneumoperitoneum and thus typically involve less blood loss. Durability, which is of little consequence in open procedures, takes on greater importance in laparoscopic procedures. With regards to a combined suction/irrigation-Kittner instrument, durability of the dissecting tip would be critical to the function of such an instrument. Without durability, multiple such instruments would need to be used through the course of one procedure, which would be cumbersome, time consuming and most importantly cost-limiting. Thus, a unique durable dissecting tip would need to be incorporated into a suction/irrigation soft tissue dissector.

Furthermore, traditional Kittners provide minimal traction or frictional surface for soft tissue dissection. Traction is necessary during soft tissue dissection to both stabilize the dissecting instrument on the surface of soft tissue as well as to allow for the dissector to more effectively dissect the tissue. Cotton/gauze based dissecting tips or instruments with smooth surfaces lack this needed traction capability. Often, surgeons have to struggle with a Kittner that routinely slips off the tissue surface, again making soft tissue dissection cumbersome and time consuming. Thus, an ideal dissecting tip would provide sufficient traction against soft tissue without causing trauma to the tissue.

In addition, the inherent “softness” of traditional Kittners also limits the “pushability” that such a device can provide. Often, surgeons need to sweep away layers of soft tissue which requires an instrument with adequate strength and pushability. Kittners utilizing soft tips have limited strength and pushability. Pushability in this context is the ability of the dissector tip to resist deformation when pressure is applied to a structure.

Yet another obstacle in fabricating an ideal suction/irrigation soft tissue dissector relates to the design of current suction/irrigation probes. Traditionally, suction/irrigation probes provide a large open-ended tip through which fluids can be aspirated and suctioned. The suctioning force is thus concentrated at the tip of the device, and often leads to the adherence of the suctioning tip to the tissue. This is not only cumbersome for the surgeon, but can also cause potential damage to the soft tissue. An ideal suctioning tip would disperse the suction force over a larger surface area, which would obviate or hinder adherence of the suctioning device to the soft tissue.

Another problem currently encountered with open ended suction irrigation probes is that these devices allow particulate material, such as blood clots, to enter the probe, only to cause obstruction downstream of the tip. While blood clots and other particulate materials do sometimes need to be removed during minimally invasive procedures, these instances are less frequent than the need to adequately suction fluids. In addition, surgeons will often employ other devices (such as forceps) to remove such materials. Thus, a suction irrigation probe that would not be prone to clogging would be a welcome improvement in the field. Additionally it would be ideal if the surgeon could filter and discriminate material being suctioned.

Some additional design issues that need to be considered in the fabrication of an ideal suction/irrigation soft tissue dissector relate to the use of such a device in providing temporary hemostasis and retraction. During minimally invasive procedures, surgeons need a fast and convenient means of applying adequate pressure to a bleeding site without causing additional trauma to the tissue. Current suction/irrigation probes are open ended thin walled tubes and thus are too aggressive and cumbersome when used to apply hemostasis. In addition, the tips of these probes tend to be somewhat sharp and can theoretically cause soft tissue trauma. Soft tissue trauma is also possible when current suction/irrigation probes are used for retraction; lack of traction at these tips also causes the device to slip off of tissue when these probes are used for retraction. Furthermore, the ability to irrigate and aspirate around the bleeding site while simultaneously holding pressure at the site would also be a welcome improvement in the field.

A final design issue that needs to be considered in the design of an ideal suction/irrigation soft tissue dissector relates to the ability of such a device to function with known suction/irrigation probes. A variety of suction/irrigation probes are available for surgical use, each with a unique tip configuration (ie. varying inner diameters, varying through-hole configurations). Thus, a soft tissue dissection tip design that would allow for use of the tip with various known suction/irrigation probes would be a welcome improvement in the field.

What is needed, therefore, are techniques for blunt dissection, retraction, application of temporary hemostasis and suction/irrigation either with a unitary device or an adjunctive dissection tip that can be coupled to known suction/irrigation probes.

### SUMMARY OF THE INVENTION

One embodiment of the present invention provides a system for the dissection of soft tissue, the system comprising: a dissection tip, the tip comprising a rigid, non-absorptive, end shell; the rigid end shell having a textured exterior surface configured to purchase soft tissue and having at least one porosity; and the dissection tip being configured to be coupled to a suction source.

Another embodiment of the present invention provides such a system wherein the rigid, blunt, non-absorptive, end shell is disposed on a tubular pin.

A further embodiment of the present invention provides such a system wherein the tip is disposed on a tubular handle.

Yet another embodiment of the present invention provides such a system wherein the tip comprises a collar receiving a distal end of the tubular handle.

A yet further embodiment of the present invention provides such a system wherein the dissection tip is integral to a suction source shaft.

Still another embodiment of the present invention provides such a system further comprising a dissection tip adaptor having a distal end disposed within the rigid, blunt, end shell, the adaptor being configured to be coupled to the suction source.

A still further embodiment of the present invention provides such a system wherein the dissection tip adaptor comprises a shaft having at least one taper, such that a proximal end of the taper has a smaller exterior diameter than a distal end of the taper and the shaft may be at least temporarily fixed within a channel of the suction source.

Even another embodiment of the present invention provides such a system further comprising an annular protrusion disposed proximal to a proximal end of the dissection tip adaptor, and at least one tension release slot transecting the annular protrusion, the annular protrusion being configured to compress upon an interior wall of the suction source.

An even further embodiment of the present invention provides such a system wherein the dissection tip adaptor further comprises a threaded portion whereby the adaptor is secured to the suction source.

Still yet another embodiment of the present invention provides such a system wherein the dissection adaptor further comprises a frictive insert whereby the adaptor is secured to the suction source.

A still yet further embodiment of the present invention provides such a system wherein the dissection adaptor comprises an adhesive whereby the adaptor is secured to the suction source.

Still even another embodiment of the present invention provides such a system wherein the end shell comprises a rigid synthetic polymer.

A still even further embodiment of the present invention provides such a system wherein the rigid synthetic polymer comprises sintered particles.

Still even yet another embodiment of the present invention provides such a system wherein the at least one porosity comprises a plurality of microporosities in the shell.

A still even yet further embodiment of the present invention provides such a system wherein the end shell comprises bonded particles.

Yet still even another embodiment of the present invention provides such a system wherein the particles are selected from the group of particles consisting of stainless steel particles, metal particles, glass particles, polypropylene particles, polyethylene particles, polytetrafluoroethylene particles, plastic particles, ceramic particles, silica, and combinations thereof.

One embodiment of the present invention provides a dissector tip mounting adaptor comprising: a dissector shell; a shell support manifold, disposed within the shell and providing suction to the shell; an insert mount, configured to be received by a suction source, wherein the insert mount is coupled to the shell support manifold and suction is directed through the adaptor to the shell support manifold.

Another embodiment of the present invention provides such a dissector tip mounting adaptor wherein the insert mount comprises a material from the group of materials consisting of metals, polymers, alloys, and combinations thereof.

A further embodiment of the present invention provides such a dissector tip mounting adaptor wherein the insert mount comprises a tapered tube having a distal end of greater outside diameter than a proximal end of the tapered tube.

Still another embodiment of the present invention provides such a dissector tip mounting adaptor further comprising an annular region of increased outside diameter proximate to the proximal end of the tube.

A still further embodiment of the present invention provides such a dissector tip mounting adaptor wherein the shell comprises a cylindrical cavity having a conic terminus, the cavity configured to receive the shell support manifold.

One embodiment of the present invention provides a system for the dissection of soft tissue, the system comprising: a dissection tip, the tip comprising a rigid, blunt, non-absorptive, end shell; the rigid blunt end shell having a textured exterior surface configured to purchase soft tissue and having at least one porosity; and the dissection tip being configured to be coupled to an irrigation source.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is an elevation view illustrating a soft tissue dissector configured in accordance with one embodiment of the present invention.

FIG. 2 is an elevation view illustrating a textured unitary soft tissue dissector configured in accordance with one embodiment of the present invention.

FIG. 3A is an elevation view illustrating a dissection tip configured in accordance with one embodiment of the present invention.

FIG. 3B is an elevation view illustrating a dissection tip configured in accordance with one embodiment of the present invention.

FIG. 3C is an elevation view illustrating a dissection tip with through holes configured in accordance with one embodiment of the present invention.

FIG. 3D is an elevation view illustrating a dissection tip with an attachment collar configured in accordance with one embodiment of the present invention.

FIG. 4A is an elevation view illustrating a dissection tip coupler configured in accordance with one embodiment of the present invention.

FIG. 4B is an elevation view illustrating a dissection tip coupler disposed within a soft tissue dissector tip configured in accordance with one embodiment of the present invention.

FIG. 4C is a cross-sectional view illustrating a dissection tip comprising microporosities within a sintered particle matrix.

FIG. 5 is an elevation view illustrating a dissection tip coupler disposed within a soft tissue dissector tip configured for insertion into a known suction irrigation probe in accordance with one embodiment of the present invention.

FIG. 6 is an elevation view illustrating a dissection tip with a check valve within a soft tissue dissector tip configured in accordance with one embodiment of the present invention.

FIG. 7 is an elevation view illustrating a dissector tip mounting adaptor configured for insertion into a known suction irrigation probe in accordance with one embodiment of the present invention.

FIG. 8 is an elevation view illustrating a dissector shaft having a tip supporting manifold configured in accordance with one embodiment of the present invention.

### DETAILED DESCRIPTION

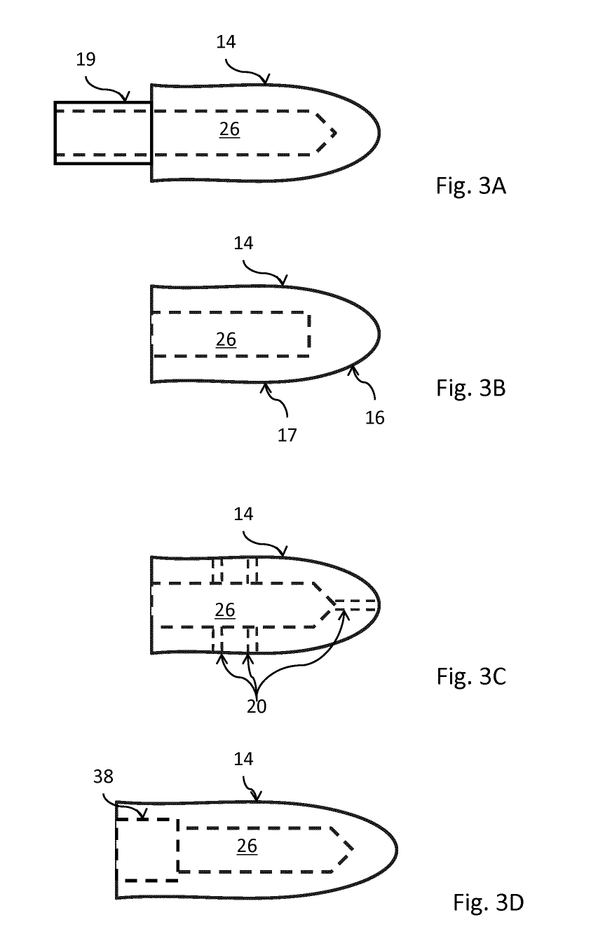

As illustrated in FIG. 3B, one embodiment of the present invention provides a rigid, non-absorbent soft tissue dissecting tip which overcomes the obstacles/problems described above by incorporating several unique features into its design. One skilled in the art will appreciate that “rigid” includes rigid and semi-rigid materials having sufficient resistance to deformation resulting in substantial retention of the material's original shape. Such a tip, intended for use with existing suction/irrigation probes or probes that are specifically either suction or irrigation sources, is configured with the durability, strength and traction needed for soft tissue dissection without causing trauma to soft tissues. In addition, the tip (while non-absorbent to maintain durability) incorporates a specific degree of porosity that allows for adequate irrigation and/or aspiration without the hindrances of adherence to adjacent tissues or clogging by particulate materials. Furthermore, the design of such a tip 14 allows for adequate pressure to be applied for hemostasis with the ability to irrigate and aspirate around the bleeding site for improved assessment of the bleeding vessel. Finally, unlike traditional Kittners, the design of the dissecting tip 14 is not soft or deformable and will not leave fibers within the surgical field. The current invention provides for a porous, blunt, atraumatic dissecting body that is rigid, non-absorbent and non-malleable, and thus provides the needed durability for a minimally invasive suction/irrigation dissecting device.

In one embodiment of the present invention, as illustrated in FIG. 3B, a soft tissue dissecting tip 14 is provided. The dissecting tip 14 comprises a rigid, blunt, non-absorptive end shell with a textured exterior surface 17. In one embodiment of the present invention the dissecting tip 14 is provided with a radius 16 at its distal end.

Such a tip or shell 14, illustrated in FIG. 3A is in one embodiment cylindrical in shape, although one skilled in the art will appreciate that other geometries may be desirable in specific situations. A coupler 19 is disposed at the proximal end of dissecting tip or shell 14, to allow for attachment of dissecting tip shell 14 to a known suction/irrigation probe. Coupler 19 is designed to have an outer diameter that will allow for insertion of the coupler into the open distal end of a known suction/irrigation probe. Coupler 19 also comprises an adequate length which will allow the coupler to occlude through-holes typically incorporated at the distal end of known suction/irrigation probes. Occlusion of these through-holes will prevent air or fluid leak at the distal end of the suction/irrigation probe when suction or irrigation is applied.

In one embodiment of the present invention, the coupler 19 is attached to a known suction/irrigation probe using a threading mechanism. One skilled in the art will appreciate that many possible methods of attachment may be provided, such as attachment with adhesives, malleable coatings, magnets, o-rings, indentations, grooves, peening, and frictive press fit. Also illustrated in FIG. 3A is a central hole having a conic tip, so as to minimize compression of particles during formation of the tip. Alternatively, embodiments without the coupler 19, as illustrated in FIG. 3B may be bonded or adhered directly to the source in a butt joint. As illustrated in FIG. 3D, a collar 38, either applied or unitary, with the shell 14 can be configured to receive the handle of the irrigator.

In one embodiment of the present invention illustrated in FIG. 3B, the tip 14 is configured with an internal cavity 26 to allow for the passage of irrigants or aspirants through the dissecting tip. In such an embodiment, the dissecting tip comprises an inherently porous material through which aspirants can be absorbed or irrigants can be dispensed. Examples of inherently porous material include, but are not limited to microporous, mesoporous and macroporous foams and ceramics, including zeolites and mesoporous silica. In an alternative embodiment of the present invention, as illustrated in FIG. 3C, through-holes 20 or macroscopic pores may be provided at different locations on the tip 14 providing channels for irrigation or aspiration when inherently non-porous materials, such as metal or plastic, are used in the fabrication of dissecting tip 14. Examples of non-porous material manufactured in such a way as to create porous tips or shells include sintered steel and other metals, sintered silica, cross linked and bonded materials, compressed materials such as plastics, ceramics, and metals. One skilled in the art will appreciate that other embodiments would include the possibility that both porous material and through-holes may be utilized to provide for enhanced aspiration and/or irrigation.

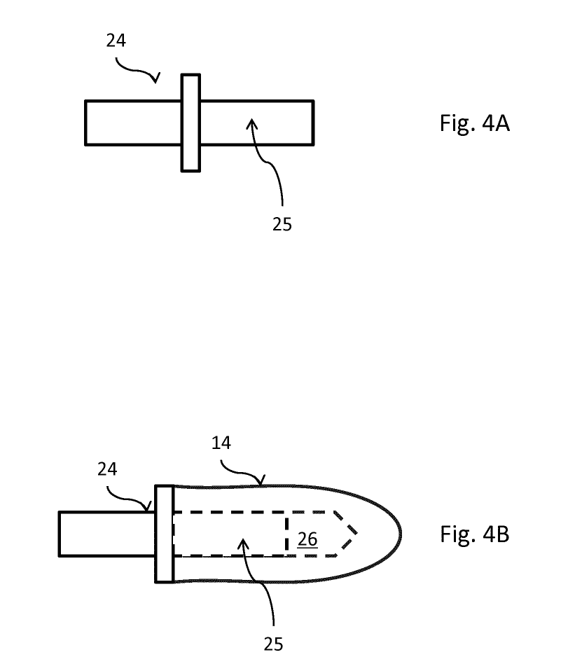

In one embodiment of the present invention, the tip 14 may be manufactured from a particle matrix. The particles of such a matrix may be manufactured from biocompatible material having suitable physical properties, including stainless steel, polypropylene, silica, ceramics, and polyethylene. In one embodiment of the present invention, such particles may be sintered or otherwise bonded. Bonding may be achieved by chemical or physical processes including, but not limited to ultrasonic welding, radio frequency welding, cross linking, irradiation, solvents, and compression. In embodiments where the sintered material lacks the structural integrity to resist breakage, an adaptor 24, as illustrated in FIGS. 4A and 4B may be configured to provide additional support to the dissector shell 23 and to couple the dissecting tip to an existing suction/irrigation probe, while providing an internal cavity for the passage of irrigants and aspirants. One skilled in the art will appreciate that the diameter of the dissecting shell 23 and the adaptor 24 is in one embodiment configured to fit smoothly within a laproscopic port and in a way that minimizes protrusions from the shaft which may catch on laparoscopic ports or tissues within the operable site. In one such embodiment, a diameter less than or equal to 5 mm is used. The dissector shell 23 can be attached to the shell support manifold 25 using any of a number of suitable techniques. In one embodiment of the present invention, an adhesive, such as cyanoacrylate is used to attach the dissector shell 23 to the shell support manifold 25. One skilled in the art will also appreciate that the shell support manifold 25 can be configured with through-holes to increase the efficiency of aspiration and irrigation through the adaptor 24.

As illustrated in FIG. 4C, in one embodiment of the present invention, the dissector shell 23 of the suction/irrigation soft tissue dissector is composed of a combination of polypropylene and polyethylene particles 27 which have been sintered to produce a specific porosity and frictional surface that is ideal for both soft tissue dissection and suction/irrigation. Furthermore, a combination of varying particle sizes are used to provide a matrix of varying pore sizes within the same tip. Such a combination of materials and particle sizes allows for the unique production of a microporous tip. In such an embodiment, as illustrated in FIG. 4B, the dissector shell 23 is configured with an internal cavity 26 with a pointed distal end. A pointed distal end provides a more uniform density of tip material at the distal end of the dissector shell 23, thereby preventing compaction and occlusion of the porosity of the dissector shell 23.

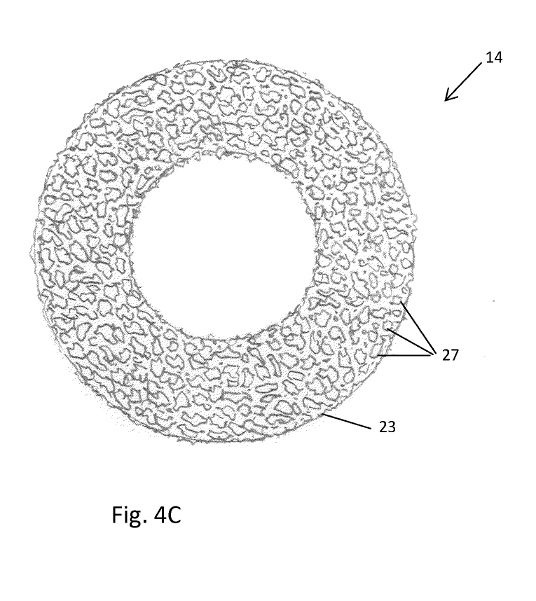

In an alternative embodiment, illustrated in FIG. 5, an adaptor mount 28 having a slight taper, in one embodiment 1°±0.5°, is provided whereby the tapered proximal end of the adaptor mount 28 may be disposed within a known suction irrigation probe (illustrated as environmental dotted lines). A tapered adaptor mount design not only provides for a secure attachment of the suction/irrigation dissection tip to a known suction/irrigation probe, but also allows for the insertion of the suction/irrigation dissection tip into known suction/irrigation probes with varying inner diameters. Thus, one suction/irrigation dissecting tip can be used with various known suction/irrigation probes. One skilled in the art will appreciate that many other possible methods of attachment may be provided between the adaptor 24 or adaptor mount 28 and known suction/irrigation probes (illustrated as environmental dotted lines), such as threaded adaptors, tapered threads, malleable coatings, adhesives, magnets, o-rings, indentations, grooves, peening, press fit, spring loaded design with a dimension that provides an appropriate interference fit, and that adaptors may be manufactured from steel, plastics, or other suitable materials.

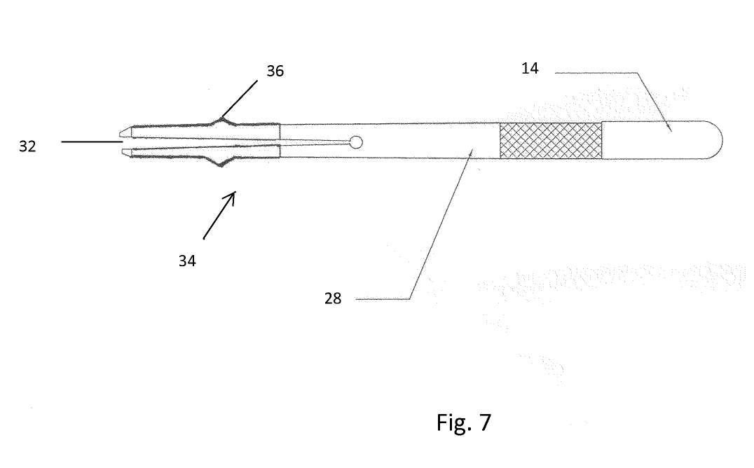

A further embodiment of the present invention, illustrated in FIG. 7 provides a tip or shell 14 and adaptor mount 28 having a longitudinal slot 32 along the length of its insertion portion 34, (the portion that mates into the known suction/irrigation probe) and a raised distal protrusion 36 around the circumference of the slot area 32 to provide a spring loaded interference fit. The distal protrusion 36 intersects with the inside diameter of the known suction/irrigation probe tube and the slot 32 enables a contraction of the insertion portion 34 outside diameter for a spring loaded interference fit.

Such an embodiment has in its design an attachment system that enables its attachment to a variety of sizes of the suction/irrigation probe's distal end tube opening without a variety of different sized adaptors. This unique design also allows such embodiments of the present invention to remain securely attached to the suction/irrigation probe even when not fully or properly installed, reducing the likelihood that the device could get dislodged during a surgery.

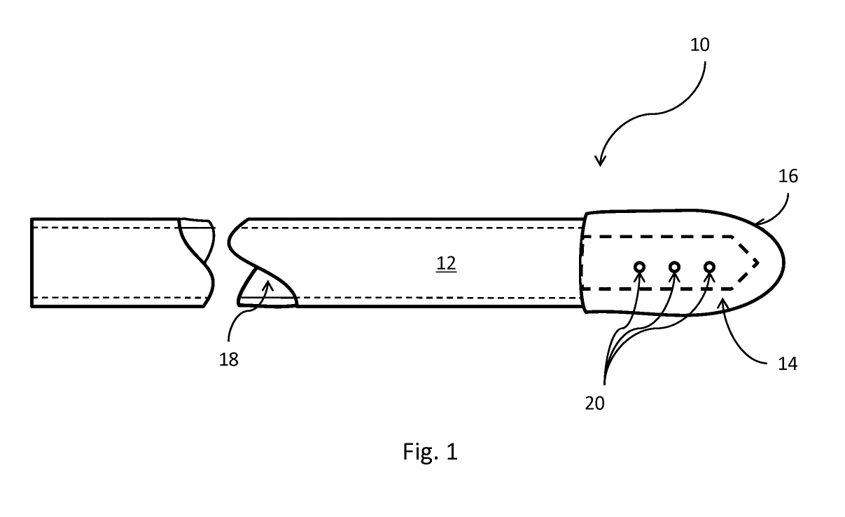

In another embodiment of the present invention, illustrated in FIG. 1, a soft tissue dissector 10 is provided. The tissue dissector has an elongate shaft 12 configured for insertion into a laparoscopic port having a distal end provided with a dissecting tip 14. The dissecting tip 14 is provided with a radius 16 at its distal end. The dissecting tip 14 is configured to allow suction or irrigation of the operable site by means of a suction or irrigation source 18 provided at the proximal end of the shaft 12. The tip 14 is, in one embodiment, configured with pores 20 disposed in the distal end of the tip and in some embodiments, pores may be disposed in the sides of the tip. Pores 20 may be macroscopic or microscopic. Alternatively, the material from which the tip 14 may be fabricated may be a porous material configured to allow suction and irrigation through the material itself. One skilled in the art will appreciate that such embodiments would include the possibility that both porous material and pores may be utilized.

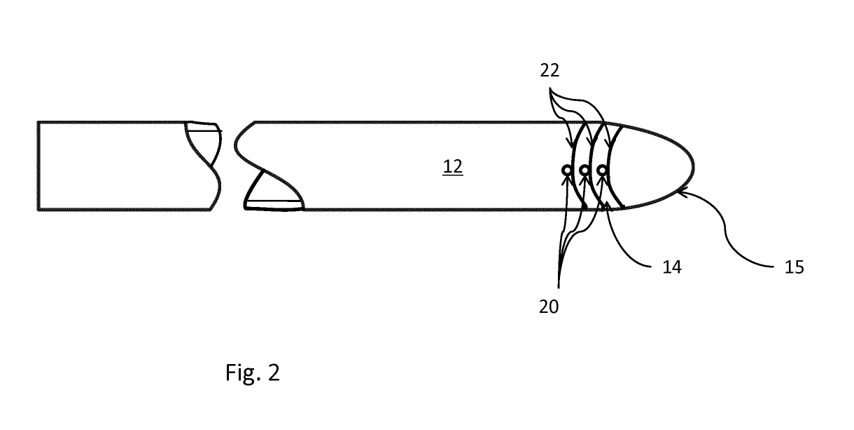

In an alternative embodiment illustrated in FIG. 2 of the present invention, the tip 14 is part of a unitary member with the shaft and may be textured 22 to allow the tip to obtain purchase on or traction against tissues that the user seeks to separate. This can be achieved, as illustrated in FIG. 2 by machining the distal end of the rigid shaft with axial grooves or splines 22; alternatively, a gnurling process can be used to provide a frictional surface to the tip 14. In alternative embodiments this texturing may be made by molding the end of the shaft 12, while in other embodiments material having a suitable porosity may be welded or fused to the shaft 12. In such a unitary embodiment, the tip 14 may be disposed with pores 20 through which fluids could be irrigated or aspirated. Finally, a rounded cap 15 with or without a central through hole would be fitted at the distal opening of the rigid tube to provide for an adequate surface for hemostasis.

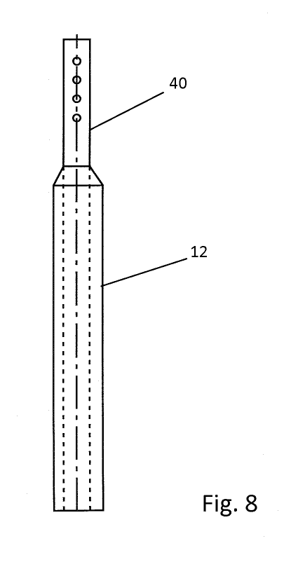

In an alternative embodiment illustrated in FIG. 8, a support manifold or pin 40 may be united with the shaft 12 and be configured to receive a textured shell or tip 14 as discussed in relation to other embodiments.

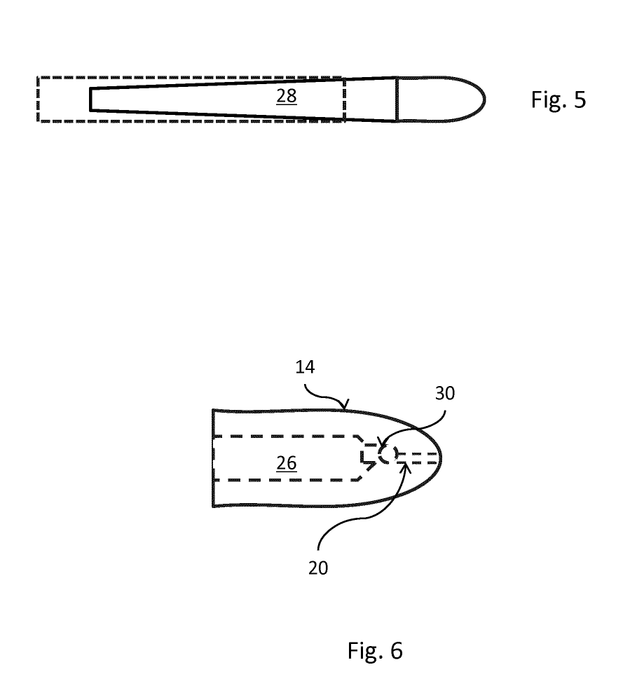

An additional embodiment, illustrated in FIG. 6 that can be incorporated into a suction/irrigation dissector tip 14 would be a one-way check valve 30. This unique feature would allow for the tip 14 to provide suction/irrigation in one flow direction, along with a diffusing or filtering like effect in the opposite flow direction. This would in turn allow surgeons the ability to regulate how aggressively they dispense fluids and the ability to discriminate while retrieving materials from the surgical site.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A system for the dissection of soft tissue, said system comprising:
a dissection tip, said tip comprising a rigid, non-absorptive, end shell;
said rigid end shell having a textured exterior surface configured to purchase soft tissue and having at least one porosity; and
said dissection tip being configured to be coupled to a suction source.

2. The system according to claim 1 wherein said rigid, non-absorptive, end shell is disposed on a tubular pin.

3. The system according to claim 1 wherein said tip is disposed on a tubular handle.

4. The system according to claim 3 wherein said tip comprises a collar receiving a distal end of said tubular handle.

5. The system according to claim 1 wherein said dissection tip is integral to a suction source shaft.

6. The system according to claim 1 further comprising a dissection tip adaptor having a distal end disposed within said rigid, end shell, said adaptor being configured to be coupled to said suction source.

7. The system according to claim 6 wherein said dissection tip adaptor comprises a shaft having at least one taper, such that a proximal end of said taper has a smaller exterior diameter than a distal end of said taper and said shaft may be at least temporarily fixed within a channel of said suction source.

8. The system according to claim 6 further comprising an annular protrusion disposed proximal to a proximal end of said dissection tip adaptor, and at least one tension release slot transecting said annular protrusion, said annular protrusion being configured to compress upon an interior wall of said suction source.

9. The system according to claim 6 wherein said dissection tip adaptor further comprises a threaded portion whereby said adaptor is secured to said suction source.

10. The system according to claim 6 wherein said dissection adaptor further comprises a frictive insert whereby said adaptor is secured to said suction source.

11. The system according to claim 6 wherein said dissection adaptor comprises an adhesive whereby said adaptor is secured to said suction source.

12. The system according to claim 1 wherein said end shell comprises a rigid synthetic polymer.

13. The system according to claim 12 wherein said rigid synthetic polymer comprises sintered particles.

14. The system according to claim 1 wherein said at least one porosity comprises a plurality of microporosities in said shell.

15. The system according to claim 1 wherein said end shell comprises bonded particles.

16. The system according to claim 13 wherein said particles are selected from the group of particles consisting of stainless steel particles, metal particles, glass particles, polypropylene particles, polyethylene particles, polytetrafluoroethylene particles, plastic particles, ceramic particles, silica, and combinations thereof.

17. A dissector tip mounting adaptor comprising:
a dissector shell;
a shell support manifold, disposed within said shell and providing suction to said shell;
an insert mount, configured to be received by a suction source, wherein said insert mount is coupled to said shell support manifold and suction is directed through said adaptor to said shell support manifold.

18. The dissector tip mounting adaptor of claim 17 wherein said insert mount comprises a material from the group of materials consisting of metals, polymers, alloys, and combinations thereof.

19. The dissector tip mounting adaptor of claim 17 wherein said insert mount comprises a tapered tube having a distal end of greater outside diameter than a proximal end of said tapered tube.

20. The dissector tip mounting adaptor of claim 17 further comprising an annular region of increased outside diameter proximate to said proximal end of said tube.

21. The dissector tip mounting adaptor of claim 20, further comprising a tension release slot transecting said annular region of increased outside diameter.

22. The dissector tip mounting adaptor of claim 17 wherein said shell comprises a cylindrical cavity having a conic terminus, said cavity configured to receive said shell support manifold.

23. A system for the dissection of soft tissue, said system comprising:
a dissection tip, said tip comprising a rigid, non-absorptive, end shell;
said rigid end shell having a textured exterior surface configured to purchase soft tissue and having at least one porosity; and
said dissection tip being configured to be coupled to a irrigation source.
